(19) United States
(12) Patent Application Publication
(10) Pub. No.: US 2006/0068025 A1
(43) Pub. Date: Mar. 30, 2006

(54) Silver microribbon composition and method of making

(75) Inventors: Yun C. Chang, Rochester, NY (US); Eric R. Schmittou, Rochester, NY (US); Peter J. Cowdery-Corvan, Webster, NY (US); Seshadri Jagannathan, Pittsford, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/953,409

(22) Filed: Sep. 29, 2004

(51) Int. Cl. A61K 33/38; H01B 1/22; H01B 1/02

(52) U.S. Cl. 424/618; 252/514

(57) ABSTRACT

This invention relates to a composition of matter comprising predominantly silver metal microribbons, wherein the microribbons are at least 1 micron in length×0.1 to 0.5 microns in width×0.05 to 0.5 microns in height. It further relates to a method of making predominantly silver microribbons comprising providing a reducible silver salt, contacting the reducible silver salt with a fogging agent to form latent image silver centers; reducing the reducible silver salt into silver metal using a reducing agent, supplying a polymer that is soluble in a non-aqueous solvent, and a non-aqueous solvent; allowing the growth of the microribbons in the presence of the polymer and non-aqueous solvent

### FIELD OF THE INVENTION

The present invention relates to a method for generating colloidal silver microribbons in non-aqueous media and the resulting compositions. The silver microribbons have many uses including antimicrobial applications and use as a conductive material.

### BACKGROUND OF THE INVENTION

Silver has long been known to be useful as a conductive material and for its antimicrobial effect. The antimicrobial properties of silver have been known for several thousand years. The general pharmacological properties of silver are summarized in “Heavy Metals”—by Stewart C. Harvey and “Antiseptics and Disinfectants: Fungicides; Ectoparasiticides”—by Stewart Harvey in The Pharmacological Basis of Therapeutics, Fifth Edition, by Louis S. Goodman and Alfred Gilman (editors), published by MacMillan Publishing Company, NY, 1975. It is now understood that the affinity of silver ion to biologically important moieties such as sulfhydryl, amino, imidazole, carboxyl and phosphate groups are primarily responsible for its antimicrobial activity.

The attachment of silver ions to one of these reactive groups on a protein results in the precipitation and denaturation of the protein. The extent of the reaction is related to the concentration of silver ions. The interaction is primarily with the proteins in the interstitial space when the silver ion concentration is low; the interaction is with the membrane proteins and intracellular species when the silver ion concentration is high. The diffusion of silver ion into mammalian tissues is self regulated by its intrinsic preference for binding to proteins as well as precipitation by the chloride ions in the environment. Thus, the very affinity of silver ion to a large number of biologically important chemical moieties (an affinity which is responsible for its action as an antimicrobial agent) is also responsible for limiting its systemic action—silver is not easily absorbed by the body. This is a primary reason for the tremendous interest in the use of silver containing species as an antimicrobial i.e. an agent capable of destroying or inhibiting the growth of microorganisms, including bacteria, yeast, fungi and algae, as well as viruses.

In addition to the affinity of silver ions to biologically relevant species, which leads to the denaturation and precipitation of proteins, it is known that some silver compounds having low ionization or dissolution ability function effectively as antiseptics. Distilled water in contact with metallic silver becomes antibacterial, even though the dissolved concentration of silver ions is less than 100 ppb. There are numerous mechanistic pathways by which this oligodynamic effect is manifested, that is, by which silver ion interferes with the basic metabolic activities of bacteria at the cellular level, thus leading to a bacteriocidal and/or bacteriostatic effect.

A detailed review of the oligodynamic effect of silver can be found in “Oligodynamic Metals” by I. B. Romans in Disinfection, Sterlization and Preservation, C. A. Lawrence and S. S. Bloek (editors), published by Lea and Fibiger (1968) and “The Oligodynamic Effect of Silver” by A. Goetz, R. L. Tracy and F. S. Harris, Jr. in Silver in Industry, Lawrence Addicks (editor), published by Reinhold Publishing Corporation, 1940. These reviews describe results that demonstrate that silver is effective as an antimicrobial agent towards a wide range of bacteria

However, it is also known that the efficacy of silver as an antimicrobial agent depends critically on the chemical and physical identity of the silver source. The silver source may be silver in the form of metal particles of varying sizes, silver as a sparingly soluble material such as silver chloride, silver as a highly soluble salt such as silver nitrate, etc. The efficiency of the silver also depends on i) the molecular identity of the active species—whether it is Ag⁺ ion or a complex species such as (AgCl₂)⁻, etc., and ii) the mechanism by which the active silver species interacts with the organism, which depends on the type of organism. Mechanisms may include, for example, adsorption to the cell wall which causes tearing; plasmolysis where the silver species penetrates the plasma membrane and binds to it; adsorption followed by the coagulation of the protoplasma; or precipitation of the protoplasmic albumin of the bacterial cell. The antibacterial efficacy of silver is determined by the nature and concentration of the active species, the type of bacteria, the surface area of the bacteria that is available to interaction with the active species, the bacterial concentration, the concentration and/or the surface area of species that could consume the active species and lower its activity, the mechanisms of deactivation and so on.

It is clear from the literature on the use of silver based materials as antibacterial agents that there is no general procedure for precipitating silver based materials and/or creating formulations of silver based materials that would be suitable for all applications. Since the efficacy of the formulations depends on so many factors, there is a need for i) a systematic process for generating the source of the desired silver species, ii) a systematic process for creating formulations silver based materials with a defined concentration of the active species; and iii) a systematic process for delivering these formulations for achieving predetermined efficacy. It is particularly a need for processes which are simple and cost effective.

There is also a need for good conductive materials. Substrates such as polymeric films or glass having an indium tin oxide (ITO) coating thereon are widely used in display devices. The requirements of such a coating are good transparency and electric conductivity. ITO coated substrates are used in applications which include touch panel devices. Touch panel devices have two opposing surfaces of the ITO films separated by spacers. Contact between the two surfaces is made when the front surface is depressed. The location of the input is decoded by an electronic interface. LCD devices include an array of transparent ITO electrodes. The electrodes are fabricated by patterning ITO coating on the substrate. In Electro-Luminescence (EL) displays electricity is converted to light. EL displays have a light-emitting layer sandwiched between two electrodes, one of which is ITO. There are a number of other applications using ITO coatings.

With the proliferation of portable electronic devices such as pagers, phones and notebook computers, ruggedness becomes an important factor in choosing a conductive coating. Since an ITO coating is relatively brittle it is highly desirable to find a more rugged conductive coating to replace ITO.

Silver is known to be an excellent conductor. If silver particles are thin enough that they do not block significant amounts of light and if particles are interconnected in a coating, it is possible to have a coating of silver particles on a substrate that exhibits high electric conductivity, good transparency and ruggedness. One particular type of silver particle that exhibits this characteristic is silver wire, which is thin, long and easy to interconnect other Ag wires in a coating.

Many methods of forming nanowires are discussed in the review article, Y. Xia and P. Yang, eds., Adv. Mater., 15 (5), 2003, but there is no mention of forming high aspect ratio forms of metallic silver by the development of silver sources such as silver halides using photographically useful developing agents under mild conditions. S. Liu, J. Yue, and A. Gedanken, Adv. Mater., 13 (9), 656 (2001) describes silver nanowires prepared from nanocrystals of AgBr (35 nm size) and a developer containing a AgNO3 component. Straight wires as long as 9 micron and 80 nm in diameter are obtained. A photographic D-72 developer produces filaments from AgBr that are 20-30 nm in diameter and that are several times longer, but not microns in length—called nanofilaments. Generally a large silver halide grain forms a mass of many nanofilaments that resembles a wad of steel wool.

C. J. Murphy and N. R. Jana, Adv. Mater. 14 (1) 80 (2002) describes a method of nanorod and nanowire formation from seeds in aqueous solution. Seeds are produced by the reduction of a soluble silver salt with borohydride in the presence of citrate as a “capping agent” to limit seed growth to 3-5 nm. Seeds and more metal salt are reduced by ascorbic acid in the presence of CTAB (a rodlike micellar template) to give rods or wires. Y. Sun and Y. Xia, Adv. Mater., 14 (11), 833 (2002) describes the formation of silver nanowires by the reduction of AgNO3 in hot ethylene glycol in presence of PVP as a “capping agent” to control the morphology. The lengths are up to 50 microns with diameters of 30-50 nm (aspect ratios up to 1000). The process requires seeds.

WO2004/019666 describes a non-continuous layer of conductive silver prepared by a photographic process. Gelatin and silver halide (AgX) are coated in a weight ratio of 0.05-0.3 on a support, exposed, and developed to give the conductive layer. Alternatively, a pattern of a nucleation agent is laid down and a silver diffusion transfer process generates the conductive layer. With gelatin and an AgX ratio of 0.4 or higher, the Ag particles do not contact each other and the resistance becomes very high. Uniformly coated and developed material has very high optical density (OD), 3.7 OD, so transparency cannot be achieved without forming a grid pattern. A pattern that is 150 micron in lines and 5 mm apart contributes about 0.1 OD. A pattern that is 1 mm lines and 10 mm apart contributes about 0.32 OD. From diffusion transfer, uniformly coated and developed material also has very high optical density—2.5 OD—so transparency is again achieved by forming a grid pattern.

U.S. Pat. No. 3,664,837 describes a light-sensitive evaporated silver halide film, which after exposure and development forms a conductive image. The areas of developed silver have high densities (low transmittance) and are quite black. Using this approach to produce a conductive, transparent layer would require forming a grid pattern of the conducting pathways, to keep the transmittance as low as possible. DE 1,938,373 describes a photographic method to produce conducting films or layers from coated silver halide emulsions. The silver halide is coated with gelatin at a gel/silver ratio of about 0.31 and at a level of about 4 g/m². The exposed coating is developed with a phenidone/hydroquinone developer with a development accelerator to give a conductive coating (resistivity of about 3-20 ohm/cm²). There is no mention of the transparency characteristics of the developed coating, however, the uniformly developed coating is expected to have low, poor transparency.

There is still needed an easy and cost effective method of forming silver microwires that are good conductors and that have low optical density.

### SUMMARY OF THE INVENTION

This invention provides a composition of matter comprising predominantly silver metal microribbons, wherein the microribbons are at least 1 micron in length×0.1 to 0.5 microns in width×0.05 to 0.5 microns in height. It further provides a method of making predominantly silver microribbons comprising
providing a reducible silver salt,
contacting the reducible silver salt with a fogging agent to form latent image silver centers;
reducing the reducible silver salt into silver metal using a reducing agent,
supplying a polymer that is soluble in a non-aqueous solvent, and a non-aqueous solvent;
allowing the growth of the microribbons in the presence of the polymer and non-aqueous solvent.

The microribbons of the invention can be prepared under lower temperature conditions. Specifically they can be prepared with a reaction temperature less than 90° C. or preferably the temperature is less than 55° C. The method is simple and cost effective and produces large sized microribbons. The developed metallic silver wires and ribbons of the invention exhibit low optical densities after formation and coating. The coated silver materials may be uniformly coated on supports, rather than requiring fabrication in grid patterns, although such patterns could be utilized if desired.

### BRIEF DESCRIPTION OF THE DRAWINGS

FIG. 1 depicts an electron micrograph of the silver microribbons made in Example 1.

### DETAILED DESCRIPTION OF THE INVENTION

The microribbons of the current invention are so named because they are flatter than conventional microwires. In general, the ratio of the width to the height of the microribbons is at least 2. Preferably the microribbons are 0.1 to 0.5 microns in width×0.05 to 0.25 microns in height, and more preferably they are 0.1 to 0.3 microns in width×0.05 to 0.15 microns in height. They are also not conventional silver particles because they are longer than a typical particle. In general, the microribbons of the invention are at least 2 times as long as they are wide. Preferably the microribbon is at least 1 micron in length, more preferably at least 10 or more microns in length and most preferably at least 15 or more microns in length.

The microribbons of the invention are predominantly silver meaning that they are greater than 50 weight % silver. Preferably they are greater than 90 weight % silver, and more preferably they are greater than 95 weight % silver. The microribbons may further comprise other metals such as copper, zinc, nickel, gold or platinum. In one preferred embodiment the microribbons further comprise copper in an amount of up to 20 weight percent.

In one embodiment the microribbon composition is made by the method of
providing a reducible silver salt,
contacting the reducible silver salt with a fogging agent to form latent image silver centers;
reducing the salt into silver metal using a reducing agent,
supplying a polymer that is soluble in a non-aqueous solvent, and a non-aqueous solvent;
allowing the growth of the microribbons in the presence of the polymer and non-aqueous solvent.

The reducible silver compounds and silver salts include silver behenate and other silver salts of long chain organic carboxylic acids. Also included are silver halides, including silver chloride, silver bromide, silver iodide, and silver halides consisting of mixtures of two or more of the halides within the silver halide crystal. Preferably the reducible silver salt is a silver halide. More preferably the silver halide is silver chloride, silver bromide, silver iodide or any mixture of chloride, bromide and iodide. Most preferably it is silver chloride. The silver halide may be in the form of silver halide grains or particles. Silver halide particles may be formed in the solvent environment described below, with the presence of polymers to stabilize the particles. The size of the silver halide particles can be changed by several factors, such as the temperature of the reaction vessel, the rates of addition of silver salt solution and halide solution, the type of polymers, the composition of halide salts, etc. such as known to those skilled in the art. General techniques for the preparation of silver halide grains may be found in “The Theory of the Photographic Process”, T. H. James, ed., 4^{th} Edition, Macmillan (1977).

The silver salt is provided to a reaction vessel and is contacted with a fogging agent. The fogging agent chemically causes the formation of silver atom clusters in the silver halide grain. The atom clusters may be known as latent image silver centers or fog centers. Fogging agents are defined as any chemical capable of generating a latent image center on the silver halide grain. Examples of fogging agents include, but are not limited to, Sn(II) compounds such as stannous chloride, borane compounds such as t-butylamine borane, and electromagnetic radiation such as visible light. The fogging agent efficiently introduces minute specks of metallic silver on the silver source. Theses specks are then developed by the action of the developing agent(s) under mild conditions to grow the silver specks into high aspect ratio wires, rods, or ribbon forms. In one embodiment the fogging agent is tin chloride. Higher temperature accelerates the fogging process and longer reaction time increases the extent of fogging reaction. Fogging agent is typically added to the silver halide emulsion at moderate temperature with vigorous stirring for up to 20 minutes

The silver atom clusters can be further enhanced by the addition of sensitizing chemicals. Sensitizing chemicals are defined as any chemical capable of increasing the efficiency of latent image formation on the silver halide grain. Such compounds are well known to those skilled in the art. Examples of sensitizing chemicals include potassium tetrachloroaurate, thiosulfate, etc. These compounds are further described in “The Theory of the Photographic Process”, T. H. James, ed., 4^{th} Edition, Macmillan (1977).

Introducing a reducing agent to the fogged silver salt particle will cause the latent image center to grow into silver wires. Reducing agents are defined as any chemical capable of reducing silver halide into silver metal. A preferred reducing agent is a photographic developing agent. Examples of reducing agents, and more particularly developing agents, include any of the useful photographic developing agents for reducing silver behenate and silver halides to metallic silver including ascorbic acid palmitate, amines, t-butylamine borane, hydroquinones, catechols, pyrogallols, p-phenylenediamines and o-phenylenediamines, p-aminophenols, complexes of Fe(II), Ti(III), and V(II), stannous chloride, hydrogen peroxide, hydroxylamines, hydrazines, hydrazides, sulfonhydrazides, ascorbic acid and its esters, alpha-hyroxycarbonyl compounds (alpha-ketols), alpha-aminocarbonyl compounds (alpha-aminoketones), hydroxytetronic acid, 1-phenyl-3-pyrazolidinone (Phenidone) and its derivatives, and other compounds as described in Chapter 11 of “The Theory of the Photographic Process”, T. H. James, ed., 4^{th} Edition, Macmillan (1977). Mixtures of developing agents can be very useful, particularly super-additive mixtures of developing agents, such as mixtures of hydroquinones with 1-phenyl-3-pyrazolidinone derivatives, and mixtures of p-aminophenols with ascorbic acid and its derivatives.

The developing agent is a significant component of this invention because a developing agent effects a more efficient formation of metallic filamentary silver, enabling milder conditions to be used. The developing agent is able to introduce its reducing electrons into metallic silver at less negative reduction potentials than does a simple reducing agent or fogging agent. This enables the growth of the silver filaments to take place without causing the formation additional developable specks on the surface of the silver source. In fact, a fogging agent is not essential to this invention. The developing agent and a source of alkalinity are sufficient to bring about the fogging and development of silver, given sufficient contact time with the silver source. Using a fogging agent makes the silver source developable more quickly. Preferred reducing agents include but are not limited to ascorbic acid esters, such as ascorbic acid palmitate and amines such as tributylamine. However, mixtures of reducing agents can be very useful.

The activity of most developing agents increases as the alkalinity of the medium increases. In aqueous systems, the alkalinity or acidity is measured by pH. Increasing pH corresponds to increasing alkalinity. In non-aqueous systems, the concept of pH does not have rigorous meaning. Nevertheless, many compounds that cause increasing pH in aqueous systems will increase the alkalinity of non-aqueous solvent systems and increase the activity of developing agents in non-aqueous solvent systems. Such sources of base or alkalinity include basic salts such as the carbonates, borates, phosphates, oxides, and hydroxides of alkali and alkaline earth metals such as lithium, sodium, potassium, magnesium, and calcium, and of tetraalkylammonium ions such as tetra-n-butylammonium. Also included are ammonia and substituted amines, such as tri-n-butylamine.

The microribbons are grown in the presence of a polymer that is soluble in a non-aqueous solvent and a non-aqueous solvent. The polymer and the solvent may be added at any point in the process as long as they are present during the growth step. Preferably they are added at the start of silver halide precipitation. In one embodiment they silver halide is formed in the presence of the polymer that is soluble in a non-aqueous solvent and the non-aqueous solvent. A non-aqueous solvent is defined as any solvent other than water. The polymer that may be utilized is any polymer that is soluble in the non-aqueous solvent. The polymer can stabilize both the silver halide particle and silver microribbon.

Non-aqueous solvents useful in the present invention include organic compounds that are liquids at the temperature used to prepare colloidal silver or the silver compound that is reduced to the colloidal silver. These solvents include aliphatic and aromatic hydrocarbon compounds such as hexane, cyclohexane, and benzene, which may be substituted with one or more alkyl groups containing from 1-4 carbon atoms. These solvents also include compounds with hydrogen-bond accepting ability. Such solvents may include one or more of the following functional groups: hydroxy groups, amino groups, ether groups, carbonyl groups, carboxylic ester groups, carboxylic amide groups, ureido groups, sulfoxide groups, sulfonyl groups, thioether groups, and nitrile groups. These solvents include alcohols, amines, ethers, ketones, aldehydes, esters, amides, ureas, urethanes, sulfoxides, sulfones, sulfonamides, sulfate esters, thioethers, phosphines, phosphite esters, and phosphate esters. Furthermore the solvents may be miscible with water such that a solvent/water mixture comprising as much as 10% by volume of water may be used as the solvent in the present invention. Preferably the solvent is a ketone. Examples of useful non-aqueous solvents include, but are not limited to, acetone, methyl ethyl ketone, acetophenone, cyclohexanone, 4-hydroxy-4-methyl-2-pentanone, isopropanol, ethylene glycol, propylene glycol, diethylene glycol, benzyl alcohol, furfuryl alcohol, glycerol, cyclohexanol, pyridine, piperidine, morpholine, triethanolamine, triisopropanolamine, dibutylether, 2-methoxyethyl ether, 1,2-diethoxyethane, tetrahydrofuran, p-dioxane, anisole, ethyl acetate, ethylene glycol diacetate, butyl acetate, gamma-butyrolactone, ethyl benzoate, N-methylpyrrolidinone, N,N-dimethylacetamide, 1,1,3,3-tetramethylurea, thiophene, tetrahydrothiophene, dimethylsulfoxide, dimethylsulfone, methanesulfonamide, diethyl sulfate, triethylphosphite, triethylphosphate, 2,2′-thiodiethanol, acetonitrile, and benzonitrile.

The polymer may be any polymer which is soluble in a non-aqueous solvent. Examples of the polymer include polyvinylbutyral. Preferably the polymer is a polyvinylbutyral or a copolymer thereof. In one preferred embodiment the polymer is polyvinylbutyral-co-vinyl alcohol co-vinyl acetate. The microribbons may be stored or sold as a composition comprising the polymer and solvent.

The invention further comprises the above method of making predominantly silver microribbons comprising
providing a reducible silver salt,
contacting the reducible silver salt with a fogging agent to form latent image silver centers;
reducing the reducible silver salt into silver metal using a reducing agent,
supplying a polymer that is soluble in a non-aqueous solvent, and a non-aqueous solvent;
allowing the growth of the microribbons in the presence of the polymer and non-aqueous solvent. A major advantage of the method is that it can be performed at a lower temperature than many of the prior art methods. The method may be performed at a temperature below 90 degrees C., more preferably below 55 degrees C. and most preferably below 35 degrees.

The microribbon composition may be concentrated or the microribbons may be isolated by filtration or other means. The microribbon composition may then be applied to an article for use, for example, as an antimicrobial or as a conductive material.

Articles having antimicrobial properties may be prepared by application of an antimicrobial compound i.e. the silver microribbons (hereafter referred to as AMC) to the surface of the article, or by embedding an AMC within the article. In most instances, bacteria or microbes may reside only at the surface of an article, and thus the AMC is applied only to the surface. The AMC may be applied by many methods such as coating, spraying, casting, blowing, extruding, etc. Typically, the AMC is dissolved or dispersed in a vehicle (such as a solvent) and a binder (such as a polymer). The vehicle serves multiple purposes including aiding the application of the antimicrobial composition via painting, spraying, coating, etc, binding the antimicrobial to that surface, and preventing the loss of antimicrobial activity due to normal wear or use. The vehicle used may be a polymer, a polymeric latex, a polymeric resin, an adhesive, or a glass or ceramic vehicle; i.e., the vehicle should comprise no more than 40% of the vehicle/antimicrobial composition mixture. Alternatively, the AMC may be mixed or compounded directly within the polymer, and the mixture subsequently melted and extruded to form a film. The film may then be attached to an article by means such as gluing or lamination. The inventive composition may be applied to the surfaces of walls, countertops, floors, furniture, consumer items, packaging, medical products such as bandages, garments, prosthetics, etc. to prevent the growth of microbes such as bacteria, mold, and yeast and to reduce the risk of the transmission of infectious disease.

This invention further relates to an antimicrobial medium, preferably a film, comprising a support and an antimicrobial layer comprising the above-described antimicrobial composition. Examples of supports useful for practice of the invention are resin-coated paper, paper, polyesters, or microporous materials such as polyethylene polymer-containing material sold by PPG Industries, Inc., Pittsburgh, Pa. under the trade name of Teslin®, Tyvek® synthetic paper (DuPont Corp.), and OPPalyte® films (Mobil Chemical Co.) and other composite films listed in U.S. Pat. No. 5,244,861. Opaque supports include plain paper, coated paper, synthetic paper, photographic paper support, melt-extrusion-coated paper, and laminated paper, such as biaxially oriented support laminates. Biaxially oriented support laminates are described in U.S. Pat. Nos. 5,853,965; 5,866,282; 5,874,205; 5,888,643; 5,888,681; 5,888,683; and 5,888,714, the disclosures of which are hereby incorporated by reference. These biaxially oriented supports include a paper base and a biaxially oriented polyolefin sheet, typically polypropylene, laminated to one or both sides of the paper base. Transparent supports include glass, cellulose derivatives, e.g., a cellulose ester, cellulose triacetate, cellulose diacetate, cellulose acetate propionate, cellulose acetate butyrate; polyesters, such as poly(ethylene terephthalate), poly(ethylene naphthalate), poly(1,4-cyclohexanedimethylene terephthalate), poly(butylene terephthalate), and copolymers thereof; polyimides; polyamides; polycarbonates; polystyrene; polyolefins, such as polyethylene or polypropylene; polysulfones; polyacrylates; polyether imides; and mixtures thereof. The papers listed above include a broad range of papers, from high end papers, such as photographic paper to low end papers, such as newsprint. Another example of supports useful for practice of the invention is fabrics such as wools, cotton, polyesters, etc.

Silver is also known to be an excellent conductor. It is possible to have a coating of silver particles on a substrate and achieve high electric conductivity, good transparency and ruggedness. Thus this invention further relates to an article comprising on the surface thereof a composition comprising predominantly silver metal microribbons, wherein the microribbons are at least 1 micron in length×0.1 to 0.5 microns in width×0.05 to 0.5 microns in height; and wherein said composition is applied to the surface in an amount and in a format suitable for conducting electrical current. The coating may be done on, for example, film or glass. The conductive coating can be used in liquid crystal display devices, touch panel devices, electro-Luminescence displays, etc.

The following examples are intended to illustrate, but not to limit, the invention.

### EXAMPLES

### Example 1

### Preparation of Microribbon

Preparing AgCl Solution in Acetone:

To a reactor charged with 20.6 g of Butvar-76, 10.5 g of lithium chloride and 500 c.c. of acetone, 54 g of solution containing 20% of silver trifluoroacetate and 80% of acetone, was added in 90 seconds under rigorous stirring. The solution was allowed to settle for 60 minutes. The supernatant was then decanted. The settled slurry is referred to as the AgCl solution for the following preparation.

Preparing Ag Microribbon:

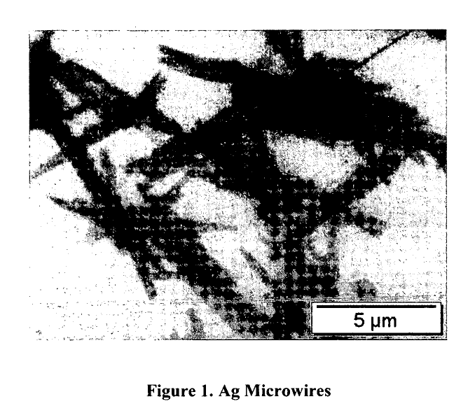

To 25 g of AgCl slurry 0.2 g of 1 percent Tin Chloride was added and the resulting mixture was left to sit at 40° C. for three minutes. Then 3 g of 0.02% potassium tetrachloroaurate, 10 g of acetone, 10 g of ascorbic acid palmitate and 7 g of tributyl amine were added. The mixture was allowed to sit at 50 C for 40 minutes. The resulting silver wires have a mean diameter of 0.5 micron and a mean length of 10 microns. A transmission electron photomicrograph of the resulting microribbons appears in FIG. 1.

The invention has been described in detail with particular reference to certain preferred embodiments thereof, but it will be understood that variations and modifications can be effected within the spirit and scope of the invention.

1. A composition of matter comprising predominantly silver metal microribbons, wherein the microribbons are at least 1 micron in length×0.1 to 0.5 microns in width×0.05 to 0.5 microns in height.

2. The composition of claim 1 wherein the microribbons are 0.1 to 0.3 microns in width×0.05 to 0.2 microns in height.

3. The composition of claim 1 wherein the microribbons are at least 10 microns in length.

4. The composition of claim 1 wherein the microribbons are at least 15 microns in length.

5. The composition of claim 2 wherein the microribbons are at least 10 microns in length.

6. The composition of claim 2 wherein the microribbons are at least 15 microns in length.

7. The composition of claim 1 wherein the microribbons are greater than 90 weight % silver.

8. The composition of claim 1 wherein the microribbons are greater than 95 weight % silver.

9. The composition of claim 1 wherein the microribbons further comprise copper, zinc, nickel, gold or platinum.

10. The composition of claim 1 wherein the microribbons further comprise copper.

11. The composition of claim 1 further comprising a polymer that is soluble in a non-aqueous solvent and a non-aqueous solvent.

12. The composition of claim 11 wherein the polymer is a polyvinylbutyral or a copolymer thereof.

13. The composition of claim 11 wherein the polymer is polyvinylbutyral-co-vinyl alcohol co-vinyl acetate.

14. The composition of claim 11 wherein the non-aqueous solvent is a ketone.

15. The composition of claim 1 made by the method of
providing a reducible silver salt,
contacting the reducible silver salt with a fogging agent to form latent image silver centers;
reducing the salt into silver metal using a reducing agent,
supplying a polymer that is soluble in a non-aqueous solvent, and a non-aqueous solvent;
allowing the growth of the microribbons in the presence of the polymer and non-aqueous solvent.

16. The composition of claim 15 wherein the reducible silver salt is a silver halide.

17. The composition of claim 15 wherein the non-aqueous solvent is a ketone.

18. The composition of claim 15 wherein the polymer is a polyvinylbutyral or a copolymer thereof.

19. The composition of claim 15 wherein the polymer is polyvinylbutyral-co-vinyl alcohol co-vinyl acetate.

20. The composition of claim 15 wherein the reducing agent is ascorbic acid palmitate or potassium tetrachloroaurate.

21. The composition of claim 15 wherein the fogging agent is tin chloride.

22. The composition of claim 15 wherein the method is performed at a temperature below 90 degrees C.

23. The composition of claim 15 wherein the method is performed at a temperature below 55 degrees C.

24. The composition of claim 1 made by the method of
providing a silver halide salt,
contacting the silver halide salt with tin chloride to form latent image silver centers;
reducing the silver halide salt into silver metal using ascorbic acid palmitate or potassium tetrachloroaurate,
supplying polyvinylbutyral or a copolymer thereof and a ketone solvent;
allowing the growth of the microribbons in the presence of the polyvinylbutyral or a copolymer thereof, and the ketone solvent; wherein the method is performed at a temperature below 55 degrees C.

25. A method of making predominantly silver microribbons comprising
providing a reducible silver salt,
contacting the reducible silver salt with a fogging agent to form latent image silver centers;
reducing the reducible silver salt into silver metal using a reducing agent,
supplying a polymer that is soluble in a non-aqueous solvent, and a non-aqueous solvent;
allowing the growth of the microribbons in the presence of the polymer and non-aqueous solvent.

26. The method of claim 25 wherein the reducible silver salt is a silver halide.

27. The method of claim 25 wherein the non-aqueous solvent is a ketone.

28. The method of claim 25 wherein the polymer is a polyvinylbutyral or a copolymer thereof.

29. The method of claim 25 wherein the polymer is polyvinylbutyral-co-vinyl alcohol co-vinyl acetate.

30. The method of claim 27 wherein the polymer is a polyvinylbutyral or a copolymer thereof.

31. The method of claim 25 wherein the reducing agent is ascorbic acid palmitate or potassium tetrachloroaurate.

32. The method of claim 25 wherein the fogging agent is tin chloride.

33. The method of claim 25 wherein the method is performed at a temperature below 90 degrees C.

34. The method of claim 25 wherein the method is performed at a temperature below 55 degrees C.

35. The method of claim 30 wherein the method is performed at a temperature below 55 degrees C.

36. The method of claim 25 made by the method of
providing a silver halide salt,
contacting the silver halide salt with tin chloride to form latent image silver centers;
reducing the silver halide into silver metal using ascorbic acid palmitate or potassium tetrachloroaurate;
supplying polyvinylbutyral or a copolymer thereof, and a ketone solvent;
allowing the growth of the microribbons in the presence of the polyvinylbutyral or a copolymer thereof, and the ketone solvent; wherein the method is performed at a temperature below 55 degrees C.

37. The method of claim 25 wherein the resulting microribbon composition is concentrated or filtered.

38. An article comprising an antimicrobial amount of a microribbon composition comprising predominantly silver metal microribbons, wherein the microribbons are at least 1 micron in length×0.1 to 0.5 microns in width×0.05 to 0.5 microns in height.

39. An article comprising on the surface thereof a composition comprising predominantly silver metal microribbons, wherein the microribbons are at least 1 micron in length×0.1 to 0.5 microns in width×0.05 to 0.5 microns in height; and wherein said composition is applied to the surface in an amount and in a format suitable for conducting electrical current.

40. A method of inhibiting the growth of microorganisms comprising contacting said microorganisms with a composition comprising predominantly silver metal microribbons, wherein the microribbons are at least 1 micron in length×0.1 to 0.5 microns in width×0.05 to 0.5 microns in height.

41. A method of conducting an electrical current comprising passing an electrical current through a device comprising a composition comprising predominantly silver metal microribbons, wherein the microribbons are at least 1 micron in length×0.1 to 0.5 microns in width×0.05 to 0.5 microns in height, wherein said composition is utilized in the device in an amount and in a format suitable for conducting electrical current.
